(19) United States
(12) Patent Application Publication
(10) Pub. No.: US 2025/0090615 A1
(43) Pub. Date: Mar. 20, 2025

(54) MULTIVITAMIN FOR AUXILIARY REGULATION OF BLOOD PRESSURE AND PREPARATION METHOD AND APPLICATION THEREOF

(71) Applicant: Changde Jizhi Biological Technology Co., Ltd, Changde (CN)

(72) Inventor: Xinqiao HE, Changde (CN)

(21) Appl. No.: 18/467,745

(22) Filed: Sep. 15, 2023

(51) Int. Cl. A61K 36/718; A61K 9/20; A61K 33/30; A61K 36/068; A61K 36/258; A61K 36/28; A61K 36/46; A61K 36/72; A61K 36/82; A61K 36/8984; A61K 36/906; A61K 45/06; A61P 9/12

(52) U.S. Cl. CPC A61K 36/718; A61K 9/20; A61K 33/30; A61K 36/068; A61K 36/258; A61K 36/28; A61K 36/46; A61K 36/72; A61K 36/82; A61K 36/8984; A61K 36/906; A61K 45/06; A61P 9/12

(57) ABSTRACT

A multivitamin for auxiliary regulation of blood pressure, a preparation method and an application thereof are provided, which relates to the field of biomedicine. Raw materials of the multivitamin include Coptis chinensis leaves, Silytum marianum, ginger, Cordyceps sinensis, Dendrobium candidum, red ginseng, Eucommia ulmoides male flower, gamma globulin, non-transgenic soybean lecithin, camellia oil, seabuckthorn seed oil, mushroom bioactive peptides, amino acids, nano-zinc oxide, and bovine bone collagen peptides. The multivitamin can significantly improve the efficacy of antihypertensive drugs and has the function of assisting in lowering blood pressure.

### STATEMENT REGARDING PRIOR DISCLOSURES BY THE INVENTOR OR A JOINT INVENTOR

Chinese publication No. CN115518150A dated Dec. 12, 2022 is a grace period inventor-originated disclosure which qualifies as an exception under 35 U.S.C. 102 (b) (1).

### TECHNICAL FIELD

The disclosure relates to the technical field of biomedicines, and more particularly to a multivitamin for auxiliary regulation of blood pressure, and a preparation method and an application thereof.

### BACKGROUND

Hypertension has become one of primary diseases threatening the health of modern people. The pathogenesis of hypertension is not yet clear, and now studies suggest that it is related to genetic and environmental factors. When the blood pressure of patients with hypertension rises to a certain extent, it will cause damage to various organs, and in severe cases, the organs will be diseased and life-threatening. Relevant researches show that the application of antihypertensive drugs to patients with hypertension can not only effectively prevent cardiovascular diseases, but also promote blood pressure to reach the standard. Therefore, in order to effectively control the cardiovascular diseases, the patient's blood pressure must be controlled first. Although hypertension is not contagious, it is a chronic disease, and it is also the main factor inducing cardiovascular and cerebrovascular diseases. Patients must take antihypertensive drugs for a long time to reduce the incidence of the cardiovascular and cerebrovascular diseases.

With the development of medicine, there are several kinds of drugs to lower blood pressure, including chemical drugs and biological drugs, etc. As a means of adjuvant therapy, traditional Chinese medicine has an important role. Therefore, it is of great significance to study and develop a new composition to improve the efficacy of the chemical drugs or the biological drugs and reduce their side effects.

### SUMMARY

Based on the above reasons, Applicant has obtained a multivitamin that can assist in regulating blood pressure through several creative labors. A crude drug of the multivitamin includes: Coptis chinensis leaves, Silytum marianum, ginger, Cordyceps sinensis, Dendrobium candidum, red ginseng, Eucommia ulmoides male flower, gamma globulin, non-transgenic soybean lecithin, camellia oil, seabuckthorn seed oil, mushroom bioactive peptides, amino acids, nano-zinc oxide, and bovine bone collagen peptides. Studies show that the multivitamin of the disclosure can significantly improve the efficacy of lowering blood pressure and has the function of assisting in lowering blood pressure.

The disclosure is realized by the following technical solutions.

Specifically, in an aspect, a multivitamin capable of assisting in regulating blood pressure includes the following raw materials: Coptis chinensis leaves, Silytum marianum, ginger, Cordyceps sinensis, Dendrobium candidum, red ginseng. Eucommia ulmoides male flower, gamma globulin, non-transgenic soybean lecithin, camellia oil, seabuckthorn seed oil, mushroom bioactive peptides, amino acids, nano-zinc oxide, and bovine bone collagen peptides.

In an embodiment, the multivitamin includes the following raw materials in parts by weight: 4-6 parts of the Coptis chinensis leaves, 5-15 parts of the Silytum marianum, 5-15 parts of the ginger, 5-15 parts of the Cordyceps sinensis, 5-15 parts of the Dendrobium candidum, 5-15 parts of the red ginseng, 5-15 parts of the Eucommia ulmoides male flower, 10-20 parts of the gamma globulin, 10-20 parts of the non-transgenic soybean lecithin, 20-40 parts of the camellia oil, 10-20 parts of the seabuckthorn seed oil, 4-6 parts of the mushroom bioactive peptides, 4-6 parts of the amino acids, 0.4-0.6 parts of the nano-zinc oxide, and 4-6 parts of the bovine bone collagen peptides.

In an embodiment, the multivitamin includes the following raw materials in parts by weight: 5 parts of the Coptis chinensis leaves, 10 parts of the Silytum marianum, 10 parts of the ginger. 10 parts of the Cordyceps sinensis, 10 parts of the Dendrobium candidum, 10 parts of the red ginseng, 10 parts of the Eucommia ulmoides male flower, 15 parts of the gamma globulin, 15 parts of the non-transgenic soybean lecithin, 30 parts of the camellia oil, 15 parts of the seabuckthorn seed oil, 5 parts of the mushroom bioactive peptides, 5 parts of the amino acids, 0.5 parts of the nano-zinc oxide, and 5 parts of the bovine bone collagen peptides.

The raw materials mentioned in the disclosure are all from Shenyao (Xianghe) Technology Co., Ltd.

The amino acids mentioned in the disclosure include glycine, alanine, valine, leucine, isoleucine, methionine, proline, tryptophan, serine, tyrosine, cysteine, phenylalanine, asparagine, glutamine, threonine, aspartic acid, glutamic acid, lysine, arginine and histidine, all of which have the same weight.

In another aspect, a preparation method of the multivitamin includes the following steps:
step 1: mixing the Coptis chinensis leaves, the Silytum marianum, the ginger, the Cordyceps sinensis, the Dendrobium candidum, the red ginseng, the Eucommia ulmoides male flower to obtain a mixture; adding water to the mixture with a water addition of 20-30 times weight of the Cordyceps sinensis; decocting the mixture added with the water for 150-200 minutes to obtain a decocted mixture; filtering the decocted mixture with an 800-mesh filter to obtain a mixed nutrient solution; putting the mixed nutrient solution in a fermentation tank and adding mucor and brown sugar into the fermentation tank with 80-100 days to obtain a fermented nutrient solution, where an adding amount of the mucor is 0.25%- 0.65% of a weight of the mixed nutrient solution, and an adding amount of the brown sugar is 5%- 8% of the weight of the mixed nutrient solution; taking out the fermented nutrient solution and adding anhydrous ethanol into the fermented nutrient solution, where an adding amount of the anhydrous ethanol is 30%- 50% of the weight of the mixed nutrient solution; and standing the fermented nutrient solution added with the anhydrous ethanol for 12-24 hours and then filtering to obtain a filtrate for later use; and
step 2: mixing the filtrate obtained in the step 1 with the gamma globulin, the non-transgenic soybean lecithin, the camellia oil, the seabuckthorn seed oil, the mushroom bioactive peptides, the amino acids, the nano-zinc oxide, and the bovine bone collagen peptides, and then stirring evenly to obtain the multivitamin.

In still another aspect, an application of the multivitamin preparation of a drug for auxiliary regulation of blood pressure is provided.

In even still another aspect, an application of the multivitamin in preparation of a drug for assisting in lowering the blood pressure is provided.

In an embodiment, an object for regulating blood pressure using the multivitamin includes human.

When the multivitamins and antihypertensive drugs are used for treatment, a small molecule water vapor therapeutic chamber (also referred to as fumigation chamber) can be used in the disclosure. The small molecule water vapor therapy chamber is equipped with gyromagnetic and far-infrared rays, affixed with 4 magnetic meteorites, and 500 grams of terahertz energy stone in the steam pot. In addition, the wavelengths of far infrared ray, terahertz wave and gyromagnetic wave which can be generated in the small molecule water vapor therapy chamber can penetrate the cell tissue, cross constantly, open the channels and collaterals, the promote blood circulation, and interact with small molecule water to clean the cell tissue.

The multivitamin extracted from various rare Chinese medicines, plants and foods contains various vitamins, trace elements, rich protein and minerals, and can quickly balance nutrition, replenish energy, enhance immunity, kill bacteria and viruses by enabling the human body to rely on its own immune system and self-healing function, repair damaged cells, and promote cell regeneration and metabolism. In addition, using small molecule steam therapy. nanoscale active lysozymes (prepared according to embodiment 1 of a Chinese patent application No.2021102481928) extracted from protein after multiple fermentations are added into the small molecule water, the patients are treated with the steam therapy to expand sweat pores, the free radicals and killed bacteria and viruses in the body are discharged, and the nanoscale active lysozymes entered the cell tissue of the body to assist the immune system to kill bacteria and viruses. By eating the multivitamin, supplementing nutrition and balancing nutrition, the immune function of the human body can be quickly restored.

### DETAILED DESCRIPTION OF EMBODIMENTS

In order to make purposes, technical solutions and advantages of the disclosure clearer, the disclosure will be further described in detail with reference to specific embodiments. It should be understood that these descriptions are only exemplary and are not intended to limit the scope of the disclosure. In addition, in the following description, descriptions of well-known structures and technologies are omitted to avoid unnecessarily confusing the concepts of the disclosure.

### Embodiment 1

A formula of raw materials of a multivitamin is: 50 grams (g) of Coptis chinensis leaves, 100 g of Silytum marianum, 100 g of ginger, 100 g of Cordyceps sinensis, 100 g of Dendrobium candidum. 100 g of red ginseng. 100 g of Eucommia ulmoides male flower, 150 g of gamma globulin, 150 g of non-transgenic soybean lecithin, 300 g of camellia oil, 150 g of seabuckthorn seed oil, 50 g of mushroom bioactive peptides, 50 g of amino acids, 5 g of nano-zinc oxide, and 50 g of bovine bone collagen peptides.

A preparation method of the multivitamin includes the following steps.

Step 1: water is added to a mixture of the Coptis chinensis leaves, the Silytum marianum, the ginger, the Cordyceps sinensis, the Dendrobium candidum, the red ginseng, the Eucommia ulmoides male flower with a water addition of 25 times weight of the Cordyceps sinensis, the mixture added with the water is decocted for 180 minutes, the decocted mixture is filtered with an 800-mesh filter to obtain a mixed nutrient solution. Then, the mixed nutrient solution is put in a fermentation tank, mucor and brown sugar are added into the fermentation tank with 90 days to obtain a fermented nutrient solution, and an adding amount of the mucor is 0.45% of a weight of the mixed nutrient solution and an adding amount of the brown sugar is 6.5% of the weight of the mixed nutrient solution. The fermented nutrient solution is taken out and added with anhydrous ethanol in an amount of 40% of the weight of the mixed nutrient solution, stood for 18 hours, and filtered to obtain a filtrate for later use.

Step 2: the filtrate obtained in the step 1 is mixed with the gamma globulin, the non-transgenic soybean lecithin, the camellia oil, the seabuckthorn seed oil, the mushroom bioactive peptides, the amino acids, the nano-zinc oxide, and the bovine bone collagen peptides, and stirred evenly to obtain the multivitamin.

### Embodiment 2

A formula of raw materials of a multivitamin is: 40 g of of Coptis chinensis leaves, 50 g of Silytum marianum, 50 g of ginger, 50 g of Cordyceps sinensis, 50 g of Dendrobium candidum, 50 g of red ginseng, 50 g of Eucommia ulmoides male flower, 100 g of gamma globulin, 100 g of non-transgenic soybean lecithin,200 g of camellia oil, 100 g of seabuckthorn seed oil, 40 g of mushroom bioactive peptides, 40 g of amino acids, 4 g of nano-zinc oxide, and 40 g of bovine bone collagen peptides.

A preparation method of the multivitamin includes the following steps.

Step 1: water is added to a mixture of the Coptis chinensis leaves, the Silytum marianum, the ginger, the Cordyceps sinensis, the Dendrobium candidum, the red ginseng, the Eucommia ulmoides male flower with a water addition of 30 times weight of the Cordyceps sinensis, the mixture added with the water is decocted for 200 minutes, the decocted mixture is filtered with an 800-mesh filter to obtain a mixed nutrient solution. Then, the mixed nutrient solution is put in a fermentation tank, mucor and brown sugar are added into the fermentation tank with 100 days to obtain a fermented nutrient solution, and an adding amount of the mucor is 0.65% of a weight of the mixed nutrient solution and an adding amount of the brown sugar is 8% of the weight of the mixed nutrient solution. The fermented nutrient solution is taken out and added with anhydrous ethanol in an amount of 50% of the weight of the mixed nutrient solution, stood for 24 hours, and filtered to obtain a filtrate for later use.

Step 2: the filtrate obtained in the step 1 is mixed with the gamma globulin, the non-transgenic soybean lecithin, the camellia oil, the seabuckthorn seed oil, the mushroom bioactive peptides, the amino acids, the nano-zinc oxide, and the bovine bone collagen peptides, and stirred evenly to obtain the multivitamin.

### Embodiment 3

A formula of raw materials of a multivitamin is: 60 g of of Coptis chinensis leaves, 150 g of Silytum marianum, 150 g of ginger, 150 g of Cordyceps sinensis, 150 g of Dendrobium candidum, 150 g of red ginseng, 150 g of Eucommia ulmoides male flower, 200 g of gamma globulin, 200 g of non-transgenic soybean lecithin, 400 g of camellia oil, 200 g of seabuckthorn seed oil, 60 g of mushroom bioactive peptides, 60 g of amino acids, 6 g of nano-zinc oxide, and 60 g of bovine bone collagen peptides.

A preparation method of the multivitamin includes the following steps.

Step 1: water is added to a mixture of the Coptis chinensis leaves, the Silytum marianum, the ginger, the Cordyceps sinensis, the Dendrobium candidum, the red ginseng, the Eucommia ulmoides male flower with a water addition of 20 times weight of the Cordyceps sinensis, the mixture added with the water is decocted for 150 minutes, the decocted mixture is filtered with an 800-mesh filter to obtain a mixed nutrient solution. Then, the mixed nutrient solution is put in a fermentation tank, mucor and brown sugar are added into the fermentation tank with 80 days to obtain a fermented nutrient solution, and an adding amount of the mucor is 0.25% of a weight of the mixed nutrient solution and an adding amount of the brown sugar is 5% of the weight of the mixed nutrient solution. The fermented nutrient solution is taken out and added with anhydrous ethanol in an amount of 30% of the weight of the mixed nutrient solution, stood for 12 hours, and filtered to obtain a filtrate for later use.

Step 2: the filtrate obtained in the step 1 is mixed with the gamma globulin, the non-transgenic soybean lecithin, the camellia oil, the seabuckthorn seed oil, the mushroom bioactive peptides, the amino acids, the nano-zinc oxide, and the bovine bone collagen peptides, and stirred evenly to obtain the multivitamin.

### Embodiment 4

A formula of raw materials of a multivitamin is: 45 g of Coptis chinensis leaves, 65 g of Silytum marianum, 65 g of ginger, 65 g of Cordyceps sinensis, 75 g of Dendrobium candidum, 65 g of red ginseng, 75 g of Eucommia ulmoides male flower, 130 g of gamma globulin, 125 g of non-transgenic soybean lecithin, 245 g of camellia oil, 135 g of seabuckthorn seed oil, 45 g of mushroom bioactive peptides, 45 g of amino acids, 5 g of nano-zinc oxide, and 45 g of bovine bone collagen peptides.

A preparation method of the multivitamin includes the following steps.

Step 1: water is added to a mixture of the Coptis chinensis leaves, the Silytum marianum, the ginger, the Cordyceps sinensis, the Dendrobium candidum, the red ginseng, the Eucommia ulmoides male flower with a water addition of 28 times weight of the Cordyceps sinensis, the mixture added with the water is decocted for 190 minutes, the decocted mixture is filtered with an 800-mesh filter to obtain a mixed nutrient solution. Then, the mixed nutrient solution is put in a fermentation tank, mucor and brown sugar are added into the fermentation tank with 95 days to obtain a fermented nutrient solution, and an adding amount of the mucor is 0.60% of a weight of the mixed nutrient solution and an adding amount of the brown sugar is 7.5% of the weight of the mixed nutrient solution. The fermented nutrient solution is taken out and added with anhydrous ethanol in an amount of 45% of the weight of the mixed nutrient solution, stood for 18 hours, and filtered to obtain a filtrate for later use.

Step 2: the filtrate obtained in the step 1 is mixed with the gamma globulin, the non-transgenic soybean lecithin, the camellia oil, the seabuckthorn seed oil, the mushroom bioactive peptides, the amino acids, the nano-zinc oxide, and the bovine bone collagen peptides, and stirred evenly to obtain the multivitamin.

### Embodiment 5

A formula of raw materials of a multivitamin is: 55 g of Coptis chinensis leaves, 140 g of Silytum marianum, 140 g of ginger, 140 g of Cordyceps sinensis, 140 g of Dendrobium candidum, 145 g of red ginseng, 140 g of Eucommia ulmoides male flower, 195 g of gamma globulin, 190 g of non-transgenic soybean lecithin, 385 g of camellia oil, 190 g of seabuckthorn seed oil, 55 g of mushroom bioactive peptides, 55 g of amino acids, 5 g of nano-zinc oxide, and 55 g of bovine bone collagen peptides.

A preparation method of the multivitamin includes the following steps.

Step 1: water is added to a mixture of the Coptis chinensis leaves, the Silytum marianum, the ginger, the Cordyceps sinensis, the Dendrobium candidum, the red ginseng, the Eucommia ulmoides male flower with a water addition of 22 times weight of the Cordyceps sinensis, the mixture added with the water is decocted for 165 minutes, the decocted mixture is filtered with an 800-mesh filter to obtain a mixed nutrient solution. Then, the mixed nutrient solution is put in a fermentation tank, mucor and brown sugar are added into the fermentation tank with 85 days to obtain a fermented nutrient solution, and an adding amount of the mucor is 0.3% of a weight of the mixed nutrient solution and an adding amount of the brown sugar is 5.5% of the weight of the mixed nutrient solution. The fermented nutrient solution is taken out and added with anhydrous ethanol in an amount of 35% of the weight of the mixed nutrient solution, stood for 15 hours, and filtered to obtain a filtrate for later use.

Step 2: the filtrate obtained in the step 1 is mixed with the gamma globulin, the non-transgenic soybean lecithin, the camellia oil, the seabuckthorn seed oil, the mushroom bioactive peptides, the amino acids, the nano-zinc oxide, and the bovine bone collagen peptides, and stirred evenly to obtain the multivitamin.

### Comparative Embodiment 1

The difference between this comparative embodiment and the embodiment 1 is only the absence of the Coptis chinensis leaves and the Dendrobium candidum, and other raw materials and the preparation method are the same as the embodiment 1.

### Comparative Embodiment 2

The difference between this comparative embodiment and the embodiment 1 is only the absence of the Silytum marianum and the ginger, and other raw materials and the preparation method are the same as the embodiment 1.

### Comparative Embodiment 3

The difference between this comparative embodiment and the embodiment 1 is only the absence of the camellia oil, the seabuckthorn seed oil and the mushroom bioactive peptides, and other raw materials and the preparation method are the same as the embodiment 1.

### Experimental Embodiment 1

### Effects on Hypertensive Rats

Experimental animals are 2-month-old spontaneously hypertensive rats.

Experimental drugs are as follows.

Experiment group 1: amlodipine besylate tablets.

Experiment group 2: amlodipine besylate tablets +the multivitamin in the embodiment 1.

Experiment group 3: amlodipine besylate tablets +the multivitamin in the embodiment 2.

Experiment group 4: amlodipine besylate tablets +the multivitamin in the embodiment 3.

Experiment group 5: amlodipine besylate tablets +the multivitamin in the embodiment 4.

Experimental group 6: amlodipine besylate tablets +the multivitamin in the embodiment 5.

Experiment group 7: amlodipine besylate tablets +the multivitamin in the comparative embodiment 1.

Experiment group 8: amlodipine besylate tablets +the multivitamin in the comparative embodiment 2.

Experiment group 9: amlodipine besylate tablets +the multivitamin in the comparative embodiment 3.

Experimental methods are as follows. Spontaneous hypertensive rats are taken and randomly divided into a model group and respective experimental groups. Specifically, in experimental group 1, amlodipine besylate tablets are given by intragastric administration at a dose of 0.05 milligrams per kilogram (mg/kg) once a day for 6 weeks. In experimental groups 2-9, amlodipine besylate tablets are given by intragastric administration at a dose of 0.05 mg/kg, and the products of the embodiments or comparative embodiments are given by intragastric administration at a dose of 600 mg/kg once a day for 6 weeks. The model group is given the same volume of distilled water once a day for 6 weeks. At 3 hours after the last administration, systolic blood pressure data are obtained on the computer through the software of noninvasive tail artery blood pressure measuring instrument. Blood pressure of each rat is measured 6-10 times each time, and the mean value is taken as the measured systolic blood pressure of the rat.

Experimental results are shown in Table 1.

TABLE 1
Effects on spontaneously hypertensive rats

| | Number | 6 weeks 3 h systolic |
|---|---|---|
| Group | of animals | blood pressure |
| Model group | 10 | 186.03 ± 3.47 |
| Experimental group 1 | 10 | 160.29 ± 2.46** |
| Experimental group 2 | 10 | 137.65 ± 2.04**# |
| Experimental group 3 | 10 | 139.89 ± 3.17**# |
| Experimental group 4 | 10 | 141.05 ± 2.18**# |
| Experimental group 5 | 10 | 140.77 ± 2.44**# |
| Experimental group 6 | 10 | 140.94 ± 1.98**# |
| Experimental group 7 | 10 | 155.26 ± 2.47 |
| Experimental group 8 | 10 | 154.78 ± 3.06 |
| Experimental group 9 | 10 | 154.06 ± 2.37 |

Note:
compared with the model group,
**P < 0.01,
*P < 0.05; and
compared with the experimental group 7,
#P < 0.05.

Experimental conclusion: the above experimental data show that the multivitamin assists amlodipine besylate to enhance the effect of lowering blood pressure, while the auxiliary effect of the comparative embodiments 1-3 (that is, removing some raw materials) disappears, the multivitamin in the comparative embodiments 1-3 in combination with amlodipine besylate has similar pharmacodynamic effect to amlodipine besylate, which fully demonstrates that the multivitamins of the embodiments have good effects of lowering blood pressure.

The above is only the illustrated embodiments of the disclosure, and it should be pointed out that those skilled in the related art can make several improvements and modifications without departing from the principle of the disclosure, and these improvements and modifications should also be regarded as the protection scope of the disclosure.

What is claimed is:

1. A multivitamin for auxiliary regulation of blood pressure, comprising the following raw materials:
Coptis chinensis leaves, Silytum marianum, ginger, Cordyceps sinensis, Dendrobium candidum, red ginseng, Eucommia ulmoides male flower, gamma globulin, non-transgenic soybean lecithin, camellia oil, seabuckthorn seed oil, mushroom bioactive peptides, amino acids, nano-zinc oxide, and bovine bone collagen peptides.

2. The multivitamin according to claim 1, comprising the following raw materials in parts by weight:
4-6 parts of the Coptis chinensis leaves, 5-15 parts of the Silytum marianum, 5-15 parts of the ginger, 5-15 parts of the Cordyceps sinensis, 5-15 parts of the Dendrobium candidum, 5-15 parts of the red ginseng, 5-15 parts of the Eucommia ulmoides male flower, 10-20 parts of the gamma globulin, 10-20 parts of the non-transgenic soybean lecithin, 20-40 parts of the camellia oil, 10-20 parts of the seabuckthorn seed oil, 4-6 parts of the mushroom bioactive peptides, 4-6 parts of the amino acids, 0.4-0.6 parts of the nano-zinc oxide, and 4-6 parts of the bovine bone collagen peptides.

3. The multivitamin as claimed in claim 1, comprising the following raw materials in parts by weight:
5 parts of the Coptis chinensis leaves, 10 parts of the Silytum marianum, 10 parts of the ginger, 10 parts of the Cordyceps sinensis, 10 parts of the Dendrobium candidum, 10 parts of the red ginseng, 10 parts of the Eucommia ulmoides male flower, 15 parts of the gamma globulin, 15 parts of the non-transgenic soybean lecithin, 30 parts of the camellia oil, 15 parts of the seabuckthorn seed oil, 5 parts of the mushroom bioactive peptides, 5 parts of the amino acids, 0.5 parts of the nano-zinc oxide, and 5 parts of the bovine bone collagen peptides.

4. The multivitamin as claimed in claim 1, wherein the multivitamin is applied to prepare a drug for the auxiliary regulation of the blood pressure.

5. The multivitamin as claimed in claim 4, wherein the multivitamin is applied to prepare a drug for assisting in lowering the blood pressure.

6. The multivitamin as claimed in claim 1, wherein an object for regulating the blood pressure comprises human.

7. The multivitamin as claimed in claim 1, wherein the multivitamin is applied to administer to a patient with hypertension in combination with an antihypertensive drug.

8. The multivitamin as claimed in claim 7, wherein the antihypertensive drug comprises
an amlodipine besylate tablet.

9. A preparation method of the multivitamin for auxiliary regulation of blood pressure as claimed in claim 1, comprising:
step 1: mixing the Coptis chinensis leaves, the Silytum marianum, the ginger, the Cordyceps sinensis, the Dendrobium candidum, the red ginseng, the Eucommia ulmoides male flower to obtain a mixture; adding water to the mixture with a water addition of 20-30 times weight of the Cordyceps sinensis; decocting the mixture added with the water for 150-200 minutes to obtain a decocted mixture; filtering the decocted mixture with an 800-mesh filter to obtain a mixed nutrient solution; putting the mixed nutrient solution in a fermentation tank and adding mucor and brown sugar into the fermentation tank with 80-100 days to obtain a fermented nutrient solution, wherein an adding amount of the mucor is 0.25%-0.65% of a weight of the mixed nutrient solution, and an adding amount of the brown sugar is 5%-8% of the weight of the mixed nutrient solution; taking out the fermented nutrient solution and adding anhydrous ethanol into the fermented nutrient solution, wherein an adding amount of the anhydrous ethanol is 30%-50% of the weight of the mixed nutrient solution; and standing the fermented nutrient solution added with the anhydrous ethanol for 12-24 hours and then filtering to obtain a filtrate for later use; and
step 2: mixing the filtrate obtained in the step 1 with the gamma globulin, the non- transgenic soybean lecithin, the camellia oil, the seabuckthorn seed oil, the mushroom bioactive peptides, the amino acids, the nano-zinc oxide, and the bovine bone collagen peptides, and then stirring evenly to obtain the multivitamin.

10. The preparation method as claimed in claim 9, wherein the multivitamin is applied to prepare a drug for the auxiliary regulation of the blood pressure.

11. The preparation method as claimed in claim 10, wherein the multivitamin is applied to prepare a drug for assisting in lowering the blood pressure.

12. The preparation method as claimed in claim 9, wherein the Coptis chinensis leaves are 4-6 parts by weight, the Silytum marianum is 5-15 parts by weight, the ginger is 5-15 parts by weight, the Cordyceps sinensis is 5-15 parts by weight, the Dendrobium candidum is 5-15 parts by weight, the red ginseng is 5-15 parts by weight, the Eucommia ulmoides male flower is 5-15 parts by weight, the gamma globulin is 10-20 parts by weight, the non-transgenic soybean lecithin is 10-20 parts by weight, the camellia oil is 20-40 parts by weight, the seabuckthorn seed oil is 10-20 parts by weight, the mushroom bioactive peptides are 4-6 parts by weight, the amino acids are 4-6 parts by weight, the nano-zinc oxide is 0.4-0.6 parts by weight, and the bovine bone collagen peptides are 4-6 parts by weight.

13. The preparation method as claimed in claim 9, wherein the multivitamin is applied to administer to a patient with hypertension in combination with an antihypertensive drug.

14. The preparation method as claimed in claim 13, wherein the antihypertensive drug comprises an amlodipine besylate tablet.

15. An application method of a multivitamin, comprising:
administering the multivitamin with an antihypertensive drug to a patient with hypertension, wherein the multivitamin comprises the following raw materials: Coptis chinensis leaves, Silytum marianum, ginger, Cordyceps sinensis, Dendrobium candidum, red ginseng, Eucommia ulmoides male flower, gamma globulin, non-transgenic soybean lecithin, camellia oil, seabuckthorn seed oil, mushroom bioactive peptides, amino acids, nano-zinc oxide, and bovine bone collagen peptides; and
assisting in lowering blood pressure of the patient with hypertension by the multivitamin.

16. The application method as claimed in claim 15, wherein the antihypertensive drug comprises an amlodipine besylate tablet.

17. The application method as claimed in claim 16, comprising:
administering the multivitamin with the antihypertensive drug to the patient with hypertension by intragastric administration.

18. The application method as claimed in claim 17, wherein a dose of the amlodipine besylate tablet of the intragastric administration is 0.05 milligrams per kilogram (mg/kg).

19. The application method as claimed in claim 18, wherein a dose of the multivitamin of the intragastric administration is 600 mg/kg.

20. The application method as claimed in claim 15, wherein the multivitamin comprises the following raw materials in parts by weight:
4-6 parts of the Coptis chinensis leaves, 5-15 parts of the Silytum marianum, 5-15 parts of ginger, 5-15 parts of the Cordyceps sinensis, 5-15 parts of the Dendrobium candidum, 5-15 parts of the red ginseng, 5-15 parts of the Eucommia ulmoides male flower, 10-20 parts of the gamma globulin, 10-20 parts of the non-transgenic soybean lecithin, 20-40 parts of the camellia oil, 10-20 parts of the seabuckthorn seed oil, 4-6 parts of the mushroom bioactive peptides, 4-6 parts of the amino acids, 0.4-0.6 parts of the nano-zinc oxide, and 4-6 parts of the bovine bone collagen peptides.
